(19) United States
(12) Patent Application Publication
(10) Pub. No.: US 2008/0280997 A1
(43) Pub. Date: Nov. 13, 2008

(54) Novel Pharmaceutical or Cosmetic Carriers Containing Cyclic Acetals

(75) Inventors: Jean-David Rodier, Villeurbanne (FR); Bruno Mahler, Lyon (FR)

Correspondence Address: HESLIN ROTHENBERG FARLEY & MESITI PC, 5 COLUMBIA CIRCLE, ALBANY, NY, 12203, US

(73) Assignee: GATTEFOSSE SAS, Saint-Priest (FR)

(21) Appl. No.: 11/568,008

(22) PCT Filed: Mar. 21, 2005
(86) PCT No.: PCT/FR05/50181

§ 371 Date: Oct. 17, 2006

(30) Foreign Application Priority Data
Apr. 21, 2004 (FR) 0404198

(51) Int. Cl. A61K 47/22

(52) U.S. Cl. 514/772

(57) ABSTRACT

The use of a cyclic acetal obtained by reacting an aliphatic aldehyde having 2 to 4 carbon atoms with a polyol having 3 to 6 carbon atoms and at least three hydroxy functions, including two on vicinal carbons or carbons having one carbon atom therebetween, as a pharmaceutical or cosmetic carrier, is disclosed.

The invention concerns novel pharmaceutical or cosmetic carriers containing cyclic acetals. It also deals with pharmaceutical or cosmetic compositions containing said carriers.

In the rest of the description and in the claims, the term “carrier” refers to a neutral substance with a variable nature to which so-called “active and preservative” agents are added, possibly dyestuffs and fragrances, the whole constituting a cosmetic or pharmaceutical composition.

The cyclic acetals are known to result from the reaction between an aldehyde and a polyol with at least two hydroxyl functions thus leading to the formation of acetal cycles having five or six atoms.

Document U.S. Pat. No. 4,031,112 describes emulsifiers used in the industry, notably in the printing field. In practice, these emulsifiers are obtained by an oxyalkylation reaction on cyclic acetals. The latter are obtained from aldehyde advantageously having between 5 and 20 carbon atoms, although formaldehyde, acetaldehyde, propionaldehyde and butyraldehyde are not expressly excluded. Moreover, the polyols used are generally polyols having 3 to 6 carbon atoms and notably glycerol or pentaerythritol. The oxyalkylation reaction is then performed in two steps in the presence of the cyclic acetal obtained, advantageously with propylene oxide.

Document GB-A-1549213 describes the use of cyclic acetals as anti-inflammatory agents in cosmetic compositions, notably in sunscreen compositions. In practice, the cyclic acetals are obtained through the reaction of an aldehyde on a diol. The aldehydes and diols which can be used for producing the anti-inflammatory agent are listed exhaustively and only certain combinations are exemplified. Among the aldehydes that can be used, the following are notably mentioned: acetaldehyde, propionaldehyde and butyraldehyde. Moreover, contrary to what is said in this document which discloses the use of diol, glycerine or glycerol, i.e. a triol, is mentioned among the usable diols. The document does not, however, expressly describe the possibility of having the glycerol react with an aldehyde having a small number of carbon atoms. Furthermore, and notably, nothing is indicated concerning the possibility of using the cyclic acetals described, as pharmaceutical or cosmetic carriers.

To the best of the Applicant's knowledge, document EP-A-0012143 describes for the first time the possibility of using certain cyclic acetals in cosmetic compositions, notably for their emollient activity. For this application it notably describes cyclic acetals resulting from the reaction between an aliphatic aldehyde having between 8 and 31 carbon atoms and a diol. Preparation No. 7, however, describes a cyclic acetal obtained from glycerol and an oxoaldehyde itself prepared with an oxo process or hydroformylation using a mixture of α-olefin in C₁₂ and C₁₄. The emollient properties of the substance thus obtained are then compared with other substances obtained from the same oxoaldehyde, but having reacted with different polyols such as ethylene glycol, 1,2-propylene glycol and pentaerythritol. As the results demonstrate, the emollient properties of the cyclic acetals obtained from glycerol and pentaerythritol are greatly inferior to those obtained with ethylene glycol or 1,2-propylene glycol.

Document U.S. Pat. No. 5,175,143 describes new cyclic acetal-based fragrances that can be used in various presentations such as bath soaps, deodorants, etc. The cyclic acetals described are obtained from cyclic aldehydes and 1-3 diol.

Document U.S. Pat. No. 5,917,059 exclusively concerns a process for preparing cyclic acetal, with no particular application. The process perfected consists in having an aldehyde having 1 to 6 carbon atoms react with a polyol having at least 2 hydroxyls. Diols and triols having 2 to 12 carbon atoms are mentioned as being advantageous, notably glycerol and trimethylolpropane.

Document EP-A-268460 describes a therapeutic composition comprising, as a penetration promoter, a dioxolane and/or a dioxane substituted by at least one R group, of which at least one is an alkyl or alkenyl group in C4 and C8. This family includes molecules with the following formula:

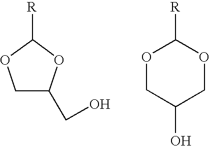

where R is an alkyl or alkenyl in C4. In practice, these molecules are obtained through a reaction between an aldehyde in C5 (pentanal or valeraldehyde) and glycerol. A mixture of 2-butyl-1,3-dioxolane-4-hydroxymethyl and 2-butyl-1,3-dioxan-5-ol is obtained. The main disadvantage of these molecules lies in the fact that they are not miscible in water, which thus limits their use as pharmaceutical or cosmetic carriers. This substance is hereinafter called BDM.

Document U.S. Pat. No. 5,686,098 describes an oestradiol-releasing patch. To improve oestradiol penetration into the skin, the patch also contains mono-isopropylidene glycerol from the reaction between acetone and glycerol. The substance obtained, sold under the name Solketal®, has the following formula:

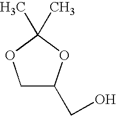

Document GB-A-2075833 describes an injectable solution containing, as its active ingredient, a mixture of trimethoprim and sulfamethoxypyridazine in a solvent, itself the result of a mixture of 4-hydroxymethyl-1,3 dioxolane and 5 hydroxy-1,3 dioxane obtained from formaldehyde.

Document FR-A-2 789 586 describes the use of certain cyclic acetals, notably Solketal®, as an absorption promoter.

In the context of the research, the Applicant observed that certain cyclic acetals obtained from specific aliphatic aldehydes and polyols presented properties, notably solvent, miscibility, and activity promoter properties, making them useful substances as pharmaceutical or cosmetic carriers.

In the rest of the description and in the claims, the expression “activity promoter” is used to express the improvement of a molecule's activity in the presence of the substances of the invention. In their absence, the active molecule in question has little or no activity in the formula being studied.

In other words, the invention concerns novel pharmaceutical or cosmetic carriers based on cyclic acetals.

These carriers are characterised in that the cyclic acetal is the product of a reaction between an aliphatic aldehyde having 2 to 4 carbon atoms and a polyol having 3 to 6 carbon atoms and at least three hydroxyl functions, at least two of which are located on vicinal carbons or are separated by a carbon.

Of course, the carrier may contain the aforementioned cyclic acetals alone or in combination with one another.

The Applicant observed that the cyclic acetals obtained through the reaction of saturated, linear or branched aldehydes having a small number of carbon atoms, in practice between 2 and 4, with, for example, a polyol having in practice between 3 and 6 carbon atoms and advantageously between 3 and 60H functions, at least two of which are located on vicinal carbons or are separated by a carbon, were useful for the desired application.

In particular, the Applicant has demonstrated that the cyclic acetals in the invention had solvent and miscibility properties better than those of similar molecules such as SOLKETAL® or the molecules described in document EP-A-268460. More generally, it was demonstrated that the cyclic acetals in the invention were soluble in many oils and alcohols used in pharmacy and cosmetics and in water, and were able to solubilise many water-soluble or insoluble active ingredients. Moreover, they are compatible with gelling agents and can be added to emulsions, microemulsions, etc. Lastly, they also have an activity promoter effect on active molecules in cosmetics or pharmaceuticals.

In an advantageous embodiment, the cyclic acetal results from the reaction between propanal (propionaldehyde) and glycerol. Because of the possible cyclisation between the hydroxyl functions of the glycerol located in positions 1 and 3 (functions separated by a carbon) or in positions 1 and 2 (vicinal) with the carbonyl function of the propionaldehyde, the reaction leads to a mixture of two molecules, respectively 2-ethyl-4-hydroxymethyl-1,3-dioxolane and 2-ethyl-1,3-dioxan-5-ol with the following formula:

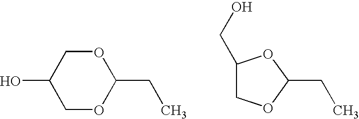

This mixture is designated as “EDM” in the rest of the description.

A mixture of the same type that also provides satisfactory results for the desired application is that of 2-propyl-4-hydroxymethyl-1,3-dioxolane with 2-propyl-1,3-dioxan-5-ol obtained by reaction between butyraldehyde and glycerol.

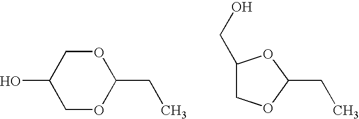

This mixture is designated as “PDM” in the rest of the description.

For these two mixtures, the Applicant observed that the mixtures enriched with one or the other of the constituents of the mixture (dioxane or dioxolane) or with a cis isomer or trans isomer of the two types of molecules, did not modify the properties obtained.

Useful properties are also obtained with 2-methyl-5-ethyl-5-hydroxymethyl-1,3-dioxane obtained by reaction between acetaldehyde and trimethylol propane (2-ethyl-2-hydroxymethyl-1,3-propanediol). The substance obtained in this case contains a mixture of cis-trans isomers of the same molecule. In fact, only one dioxane cycle is obtained with this process. In this case, the hydroxyl functions are separated by a carbon atom. In the rest of the description, the substance obtained is designated as “ETP” and has the following formula:

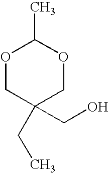

The processes for preparing cyclic acetals are perfectly well known and are of two types, respectively, a process with a solvent, notably toluene, and a process without solvents. These processes are described below.

The invention, of course, concerns a pharmaceutical or cosmetic composition containing the aforementioned carrier.

Notably, the cyclic acetals in the invention can be used for their solubilising properties on active ingredients that are little or not at all soluble in water or oil, and as an activity promoter probably as the result of an absorption promoter effect.

The composition containing the cyclic acetals in the invention may come in all of the pharmaceutical forms normally used for a topical application on the skin or hair, notably in the form of an aqueous solution, an oil-in-water or water-in-oil or multiple emulsion, a silicone emulsion, a microemulsion or nanoemulsion, or an aqueous gel.

This composition may be more or less fluid and have an appearance that is, amongst others, a white or coloured cream, an ointment, a milk, a lotion, a serum or a gel.

The composition may contain the usual adjuvants used in the cosmetic and dermatological fields, such as oil phases, emulsifiers and co-emulsifiers, hydrophilic or lipophilic gelling agents, hydrophilic or lipophilic active ingredients, preservatives, antioxidants, solvents, fragrances, fillers, hydrophilic and lipophilic filters, dyestuffs, neutralising agents, propenetrating agents and polymers.

The quantities of these various adjuvants are those conventionally used in the fields in question and, for example, between 0.01 and 30% of the total weight of the composition. These adjuvants, depending on their nature, may be added in the oil phase or in the aqueous phase.

The oil phases that can be used in the invention include vegetable oils and fats, mineral oils, oils of animal origin (such as lanolin), synthetic oils (for example, isopropyl myristate, octyldodecyl, isostearyl isostearate, decyl oleate or isopropyl palmitate), silicone oils (cyclomethicone, dimethicone) and fluorinated oils. The following can be used as fats: fatty alcohols, fatty acids, waxes and gums and notably silicone gums.

As emulsifiers and co-emulsifiers that can be used, we can mention, for example, polyglycerol fatty acid esters, sucrose fatty acid esters, sorbitan fatty acid esters, oxyethylenated sorbitan fatty acid esters, PEG fatty acid ethers, glycerol fatty acid esters, alkyl sulphates, alkyl ether sulphates, alkyl phosphates, alkyl polyglucosides and silicone emulsifiers.

As hydrophilic gelling agents, we can notably mention carboxyvinyl polymers (carbomer), acrylic copolymers such as acrylate/alkylacrylate copolymers, polyacrylamides, polysaccharides such as xanthan gum, guar gum, natural gums such as cellulose gum and derivatives and clays.

As lipophilic gelling agents we can mention modified clays such as bentones, fatty acid metal salts, hydrophobic silica and ethylcellulose.

As active ingredients we can notably use depigmenting agents, emollients, moisturisers, antiseborrheic agents, antiacne agents, keratolytic and/or desquamative agents, anti-wrinkle agents and tensors, draining agents, anti-irritation agents, soothing agents, slimming products such as xanthic bases (caffeine), vitamins and their combinations, matting agents, anti-ageing ingredients such as retinol, anti-wrinkle agents, and essential oils.

In case of incompatibility between them, the aforementioned active ingredients may be added in spheroids, notably ionic or non-ionic capsules and/or nanoparticles (nanocapsules and/or nanospheres), so as to isolate them from each other in the composition.

Among the preservatives that can be used, we can mention benzoic acid, its salts and its esters; sorbic acid and its salts; parabens, their salts and esters; triclosan; imidazolidinyl urea; phenoxyethanol; DMDM hydantoin; diazolidinyl urea; chlorphenesin.

Among the antioxidants that can be used, we can mention BHA, BHT, TBHQ, propyl gallate, tocopherols and their esters, tocotrienols, ascorbyl palmitate, extracts of rosemary, extract of green tea, chlorogenic acid, beta-carotene, flavonoids, chelating agents such as EDTA and its salts, and citric acid.

Solvents that can be used include water, ethanol, glycerine, propylene glycol, butylene glycol and sorbitol.

Fillers that can be used include talc, kaolin, mica, serecite, magnesium carbonate, aluminium silicate, magnesium silicate and organic powders such as nylon.

Filters that can be used include conventionally used UVA and UVB filters such as benzophenone-3, butyl methoxydibenzoyl methane, octocrylene, octyl methoxycinnamate, 4-methylbenzylidene camphor, octyl salicylate, titanium dioxide and zinc oxide in their micrometric and nanometric forms.

Dyestuffs that can be used include lipophilic dyes, hydrophilic dyes, pigments and mother-of-pearl commonly used in cosmetic or dermatological compositions, and their combinations.

Neutralising agents that can be used include soda, triethanolamine, aminomethyl propanol and potassium hydroxide.

Propenetrating agents that can be used include alcohols and glycols (ethanol, propylene glycol), ethoxydiglycol, fatty acid alcohols (oleic acid), fatty acid esters and dimethyl isosorbide.

The composition containing a carrier from the invention may be used as a care product, a cleaning product and/or a skin make-up product, as a sunscreen product or as a hair product, for example as a shampoo or conditioner.

The invention and the advantages it provides will become clearer with the following examples of its embodiment backed up by the appended figures.

### EXAMPLE 1

### EDM Synthesis Process with and without Solvents

1/ Synthesis with an Azeotropic Solvent

92 g (1 mole) glycerol, 0.45 g p-toluenesulfonic acid and 150 ml cyclohexane are mixed in a stirring reactor. 69 g (1.2 mole) propanal is poured, drop by drop, using a pressure compensating device. After adding aldehyde, the reaction medium is heated to at least 69° C., temperature of the water/cyclohexane azeotrope. The water is separated from the cyclohexane with a Dean-Stark apparatus. Heating is maintained until the theoretical quantity of water has been retrieved. Cyclohexane evaporates under low pressure and the reaction substance (EDM) is purified by low-pressure distillation using a Vigreux distilling column. The mass yield from the reaction is 70% and the substance has a level of purity greater than 99%. Analyses with NMR and gas chromatography coupled with a mass spectrometer (GC-MS) confirm that the substance obtained is a mixture of 2-ethyl-1,3-dioxolane-4-hydroxymethyl and 2-ethyl-1,3-dioxan-5-ol.

2/ Synthesis without Solvents

92 g glycerol (1 mole) and 0.45 g p-toluenesulfonic acid are placed in a stirring reactor. 58 g (1 mole) propanal is added, drop-by-drop, in 1 hour, using a pressure compensating device. The mixture is heated to 46° C. for 5 hours. The reaction water is eliminated by low-pressure distillation (50 mbar). It is retrieved at between 30 and 35° C. in the reactor head. The EDM is then purified by low-pressure distillation (10 mBar) using a Vigreux distilling column. The mass yield from the reaction is 70% and the substance has a level of purity greater than 99%. The NMR and GC-MS analyses confirm that the substance obtained is a mixture of 2-ethyl-1,3-dioxolane-4-hydroxymethyl and 2-ethyl-1,3-dioxan-5-ol.

### EXAMPLE 2

### Solvent Properties of EDM, PDM and ETP

1/ Miscibility in Water

The BDM molecule is produced following the same process as that used in example 1 (without solvents), using pentanal and glycerol. The substance obtained is confirmed by GC-MS analysis. Its water solubility is approximately 2 g in 100 g water. Beyond that, turbidity appears.

| Substance studied | Water |
|---|---|
| Glycerol formal\textcircled{1} | Miscible |
| Solketal ® | Miscible |
| EDM | Miscible |
| PDM | Miscible |
| BDM | Non-miscible |
| ETP | Miscible |

\textcircled{1}Cyclic acetal resulting from the reaction of formaldehyde on glycerol

### Conclusion:

As the above table shows, the molecules in the invention are miscible in water, unlike BDM.

2/ Solubility in Oils

Solubility (in g of solvent solubilised in 100
g of oil or ester before turbidity appears)

| | Medium-chain | | |
|---|---|---|---|
| Solvent | triglyceride | Sunflower | Octyldodecanol |
| studied | (MCT) | oil | myristate (ODM) |
| Glycerol | <12 | 12 | <12 |
| formal\textcircled{1} | | | |
| Solketal ® | >100 | 37 | 25 |
| EDM | >100 | >100 | 50 |
| PDM | >100 | >100 | 100 |
| ETP | >100 | >100 | 100 |

\textcircled{1}Cyclic acetal resulting from the reaction of formaldehyde on glycerol

### Conclusion:

As the above table shows, while remaining miscible in water, the cyclic acetals in the invention have better solvent power than SOLKETAL® or glycerol formal.

This property demonstrates their better compatibility with lipophilic components for cosmetic and pharmaceutical use and thus their wider range of uses.

3/ Solubility of EDM in Oils and Alcohols

The solubility of EDM is studied in various oils, alcohols and water. The solubility results are expressed in g of EDM incorporable into 100 g of oil or ester before turbidity appears.

| | | | EDM |
|---|---|---|---|
| Esters | INCI name | Supplier | mass |
| Arlamol ® E | PPG-15 Stearyl Ether | Uniqema | >100 |
| DPPG | Propylene glycol dipelargonate | Gattefossé | >100 |
| IPM | Isopropyl myristate | | >100 |
| Kernel oil | Apricot kernel oil | | >100 |
| Castor oil | Castor oil | | >100 |
| Tegosoft ® TN | C12/C15 alkyl benzoate | Degussa | >100 |
| Labrafac ® CC | Caprylic/capric triglyceride | Gattefossé | >100 |

| Hydrophilic solvents | INCI name | EDM mass |
|---|---|---|
| Butylene Glycol | Butylene Glycol | >100 |
| Water | Water | >100 |
| Glycerine | Glycerine | >100 |
| PEG-300 | PEG-300 | >100 |
| Propylene Glycol | Propylene Glycol | >100 |

### Conclusion:

EDM is highly soluble in many esters, in hydrophilic solvents and in vegetable oils.

4/ Solubilisation of Filters with ETP, EDM and PDM

The solubility of two organic UV filters (benzophenone-3 et 1,2-butyl methoxydibenzoylmethane) are assessed in ETP, EDM and PDM.

### Protocol:

homogenise the mixture for 30 seconds in a vortex mixer,
apply ultrasounds for 15 minutes (repeat if the filter is not miscible up to a maximum of 60 minutes),
for the following percentages, an identical quantity of UV filter is always added to a set initial quantity of solvent.

Solubility is expressed in g of filter soluble in 100 g of final solution.

| Solvents | Benzophenone-3 | 1,2-butyl methoxydibenzoylmethane |
|---|---|---|
| SOLKETAL ® | 18 | 5 |
| ETP | 35 | 5 |
| EDM | >40 | >32 |
| PDM | 18 | 22 |

### Conclusion:

The molecules tested are better filter solubilising agents than SOLKETAL®.

5/ Solubilisation of Active Ingredients in EDM

The solubility of the various active ingredients in EDM was studied. It is expressed in g of active ingredient soluble in 100 ml of EDM.

| Active | | Mass of active |
|---|---|---|
| ingredients | INCI name | ingredient (g) |
| Vitamin E | Tocopherol | >100 |
| Bisabolol nat. ⁽¹⁾ | Bisabolol | >100 |
| Menthol | Menthol | >100 |
| Lavandin EO | Lavandin oil | >100 |
| Eucalyptus EO | Eucalyptus globulus leaf oil | >100 |
| Parsol MCX ⁽²⁾ | Ethylhexyl methoxycinnamate | >100 |
| Vitamin A | Retinal | >100 |

⁽¹⁾ BASF
⁽²⁾ ROCHE

### Conclusion:

The cosmetic active ingredients are highly soluble in the solvent EDM.

### EXAMPLE 3

### Solubilisation of Non-Water Soluble Active Ingredients Assisted by EDM

Five hydrophobic active ingredients were solubilised in water using EDM.

The following table indicates what mass of solvent (g) must be added to solubilise 1 g of a mixture comprising 98% water and 2% active ingredient.

The smaller the quantity of solvent to be added, the greater its solubilising power.

| Active | | Mass of | Arlasolve |
|---|---|---|---|
| ingredients | INCI name | EDM (g) | DMI ⁽²⁾ |
| Vitamin E | Tocopherol | 2.0 | 5.0 |
| Bisabolol nat. | Bisabolol | 0.9 | 2.3 |
| Menthol | Menthol | 0.7 | 1.7 |
| HE eucalyptus | Eucalyptus globulus | 1 | 2.2 |
| | leaf oil | | |
| Eusolex 4360 ⁽¹⁾ | Benzophenone 3 | 1.7 | 2.3 |

⁽¹⁾ MERCK
⁽²⁾ Uniqema

### Conclusion:

EDM has a solubilising power on hydrophobic active ingredients in water. It provides better performances than Arlasolve DMI (Dimethyl Isosorbide), another solvent used in the cosmetics market.

### EXAMPLE 4

### Stability of the pH of an EDM Solution

The stability of solutions at different pHs after adding 5% EDM was studied.

### 1. Buffer Solutions

The different solutions were prepared:
using citric acid and Na₂HPO₄ for pHs between 3 and 7,
using boric acid, KCl and NaOH for pHs between 8 and 10.

### 2. Results

| | | | | | | | | |
|---|---|---|---|---|---|---|---|---|
| Initial pH | 3.1 | 4.25 | 5.1 | 6.15 | 7.15 | 8.0 | 9.0 | 10.0 |
| pH after | 3.15 | 4.3 | 5.15 | 6.2 | 7.2 | 8.0 | 9.1 | 10.0 |
| adding EDM | | | | | | | | |
| pH at 1 | 3 | 4.2 | 5.1 | 6.1 | 7.1 | 8.0 | 9.0 | 10.0 |
| month | | | | | | | | |

### Conclusion:

The variations observed are approximately 0.1 pH units, which is not significant. Consequently, EDM has no influence on the pH of buffer solutions from 3 to 10.

### EXAMPLE 5

### Compatibility of EDM with the Components of a Cosmetic Formula

1. Compatibility with Gelling Agents

We tested the influence of 5% EDM in gels produced using gelling agents with different chemical natures.

| Gelling agent | | | |
|---|---|---|---|
| Commercial name | INCI name | Supplier | Viscosity |
| Blanose 7HF | Cellulose Gum | Aqualon | Small |
| Jaguar HP 105 | Hydroxypropyl guar | Rhodia | variation |
| Keltrol T | Xanthan Gum | Kelco | |
| Natrosol 250HX | Hydroxyethyl cellulose | Aqualon | |
| Avicel RC591 | Microcrystalline | FMC | Drop by |
| | Cellulose, Cellulose Gum | | 10 to 30% |
| Carbopol | Acrylates/C10-30 Alkyl | Noveon | |
| ETD 2020 | acrylate crosspolymer | | |
| Carbopol | Carbomer | Noveon | |
| Ultrez 10 | | | |
| Novemer EC-1 | Acrylates/Acrylamide | Noveon | Drop >30% |
| | copolymer mineral oil, | | |
| | polysorbate-85 | | |
| Sepigel 305 | Polyacrylamide, C13-14 | Seppic | |
| | Isoparaffin, laureth-7 | | |

### Conclusion:

EDM presents good compatibility with the gelling agents of the different families tested. In fact, none of the gels formulated is broken by adding EDM.

### 2. Compatibility in Emulsion

2.1—O/W Emulsion

| | Formula 1 | Formula 2 | Supplier | INCI name |
|---|---|---|---|---|
| Emulium 22 | 6 | 6 | Gattefossé | Tribehenin PEG-20 |
| | | | | Esters |
| ODM | 15 | 15 | Gattefossé | Octyldodecyl |
| | | | | Myristate |
| Phenonip | 0.5 | 0.5 | Nipa | Phenoxyethanol, |
| | | | | Methylparaben, |
| | | | | Ethylparaben, |
| | | | | Butylparaben, |
| | | | | Propylparaben, |
| | | | | Isobutylparaben |
| Demineralised water | 78.5 | 73.5 | | Water |
| EDM | — | 5 | | |
| Viscosity, Brookfield | 96,000 | 82,000 | | |
| LVDII (cP) 1 month | | | | |
| Stability at AT | 1 month OK | 1 month OK | | |
| Stability at 40° C. | 1 month OK | 1 month OK | | |
| Stability at 50° C. | 1 month OK | 1 month OK | | |

### Conclusion:

Adding EDM to the emulsion does not significantly modify viscosity and stability.

2.2—Emulsion and Bi-Gel

Bi-gel is a surfactant-free emulsion.

| | Formula | Supplier | INCI name |
|---|---|---|---|
| Demineralised | 74 | | Water |
| water | | | |
| Glydant Plus | 0.5 | Lonza | DMDM Hydantoin, |
| Liquid | | | Iodopropynyl |
| | | | Butylcarbamate |
| Ultrez 10 | 0.25 | Noveon | Carbomer |
| AMP (sol. at 50%) | 0.25 | Angus | Aminomethyl Propanol |
| EDM | 5 | | |
| Emulfree CBG | 5 | Gattefossé | Isostearyl Alcohol, |
| | | | Butylene Glycol |
| | | | Cocoate, Ethylcellulose |
| Arlamol HD | 15 | Uniqema | Isohexadecane |
| Appearance | Homogenous | | |
| | Bi-gel | | |
| pH at 1 month | 5.3 | | |
| Viscosity, | 40000 | | |
| Brookfield | | | |
| LVDII (cP) | | | |
| 1 month | | | |
| Stability at AT | 1 month OK | | |
| Stability at 40° C. | 1 month OK | | |
| Stability at 50° C. | 1 month OK | | |

### Conclusion:

The emulsion obtained is perfect. EDM is compatible with the components of a Bi-gel emulsion.

### 3. Compatibility in Microemulsion

| | Formula | Supplier | INCI name |
|---|---|---|---|
| LAS | 23.33 | Gattefossé | PEG-8 Caprylic/Capric Glycerides |
| Plurol | 11.67 | Gattefossé | Polyglyceryl-3 Diisostearate |
| diisostearic | | | |
| Isopar L | 50 | Exxon | C11-13 Isoparaffin |
| Phenonip | 0.5 | Nipa | Phenoxyethanol, Methylparaben, |
| | | | Ethylparaben, Butylparaben, |
| | | | Propylparaben, Isobutylparaben |
| Demineralised | 10 | | Water |
| water | | | |
| EDM | 5 | | |
| Stability at AT | OK | | |

### Conclusion:

EDM can be formulated in microemulsions.

### EXAMPLE 6

### In Vitro Assessment of the Capacity of EDM to Increase the Depigmenting Activity of Kojic Acid (Activity Promoter Effect of the Molecule)

We sought to assess, in vitro, the capacity of EDM to increase the level of effectiveness of kojic acid on pigmented human epidermises reconstructed in vitro.
The results obtained with several emulsions were compared: reference emulsion ALE 1833/A: carrier formula, without kojic acid
ALE 1833/B: formula with 2% kojic acid
ALE 1833/C: formula with 2% kojic acid and 5% EDM
Positive control: MelanexDuo® Cream

### ALE 1833A Cream

| Ingredients | INCI designation | % W/W |
|---|---|---|
| Phase 1 | | |
| EMULIUM DELTA (1) | Cetyl alcohol (and) glyceryl | 6.00 |
| | stearate (and) PEG-75 | |
| | stearate (and) CETETH-20 | |
| | (and) STEARETH-20 | |
| SILKFLO 364 NF (2) | Polydecene | 15.00 |
| GLYDANT PLUS LIQUID (3) | DMDM hydantoin (and) | 0.40 |
| | iodopropynyl butylcarbamate | |
| Phase 2 | | |
| Demineralised water | Water | 78.30 |
| CARBOPOL ULTREZ 10 (4) | Carbomer | 0.15 |
| Phase 3 | | |
| AMP-95 (50% SOL.) (5) | Aminomethyl propanol | 0.15 |
| | | 100.00 |

(1) Gattefossé S.A./
(2) Amoco Chemical/
(3) Lonza/
(4) Noveon/
(5) Angus

### ALE 1833B Cream

| Ingredients | INCI designation | % W/W |
|---|---|---|
| Phase 1 | | |
| EMULIUM DELTA (1) | Cetyl alcohol (and) glyceryl | 6.00 |
| | stearate (and) PEG-75 | |
| | stearate (and) CETETH-20 | |
| | (and) STEARETH-20 | |
| SILKFLO 364 NF (2) | Polydecene | 15.00 |
| GLYDANT PLUS LIQUID (3) | DMDM hydantoin (and) | 0.40 |
| | iodopropynyl butylcarbamate | |
| Phase 2 | | |
| Demineralised water | Water | 66.30 |
| CARBOPOL ULTREZ 10 (4) | Carbomer | 0.15 |
| Phase 3 | | |
| AMP-95 (50% SOL.) (5) | Aminomethyl propanol | 0.15 |
| Phase 4 | | |
| KOJIC ACID (6) | KOJIC ACID | 2.00 |
| Demineralised water | Water | 10.00 |
| | | 100.00 |

(1) Gattefossé S.A./
(2) Amoco Chemical/
(3) Lonza/
(4) Noveon/
(5) Angus/
(6) Provided

### ALE 1833C Cream

| Ingredients | INCI designation | % W/W |
|---|---|---|
| Phase 1 | | |
| EMULIUM DELTA (1) | Cetyl alcohol (and) glyceryl | 6.00 |
| | stearate (and) PEG-75 | |
| | stearate (and) CETETH-20 | |
| | (and) STEARETH-20 | |
| SILKFLO 364 NF (2) | Polydecene | 15.00 |
| GLYDANT PLUS LIQUID (3) | DMDM hydantoin (and) | 0.40 |
| | iodopropynyl butylcarbamate | |
| Phase 2 | | |
| Demineralised water | Water | 61.30 |
| CARBOPOL ULTREZ 10 (4) | Carbomer | 0.15 |
| Phase 3 | | |
| AMP-95 (50% SOL.) (5) | Aminomethyl propanol | 0.15 |
| Phase 4 | | |
| EDM lot 02-02 (1) | | 5.00 |
| KOJIC ACID (6) | KOJIC ACID | 2.00 |
| Demineralised water | Water | 10.00 |
| | | 100.00 |

(1) Gattefossé S.A./
(2) Amoco Chemical/
(3) Lonza/
(4) Noveon/
(5) Angus/
(6) Provided

### General Methodology

The study is based on the assessment of the degree of pigmentation in pigmented human epidermises after treatment with the different test substances.

The depigmenting activity of the different formulas is assessed by measuring the level of melanin produced by a cell suspension.

The level of melanin is determined by measuring optical density at 405 nm of cell extracts obtained from epidermises treated with the test substances.

The depigmenting activity is then calculated using the following formula:

D.A%=[C_{Mel(carrier)}−C_{Mel(substance)}/C_{Mel(carrier)}]×100

C_{Mel (carrier)}: level of melanin in the cell extracts obtained from epidermises treated with carrier formula ALE 1833/A
C_{Mel (substance)}: level of melanin in the cell extracts obtained from epidermises treated with carrier formula ALE 1833/B or ALE 1833/C

The test is performed in triplicate.

A preliminary cytotoxicity study is performed to verify the viability of the cell in the presence of the experimental components:
Application on D0 (=day of the start of the study) and D1 (=D0+24 hours) of formulas ALE 1833/A, B and C, at 5 mg/cm² on the stratum corneum of the non-pigmented human epidermises. Incubation of the epidermises at 37° C.
Assessment of the viability of the tissues using an MTT test (3-(4,5-dimethylthiazol-2-yl)-2,5-diphenyltetrazolium bromide) on D+2.

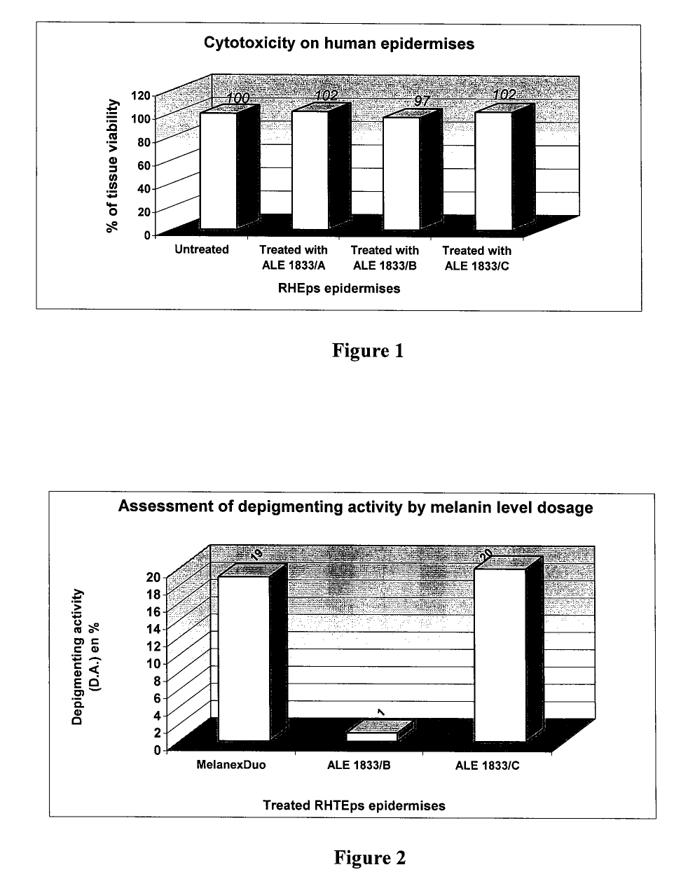

The results obtained are summarised in FIG. 1:

The depigmenting activity of the study substances is assessed as follows:
Application, on D0, D1, D2, D3 and D6, of formulas ALE 1833/A, B and C and of MelanexDuo® Cream, at 2.5 mg/cm² on the stratum corneum of pigmented human epidermises. Incubation of the epidermises at 37° C. in an air-CO₂ incubator.
Determination of the level of melanin at D7, 24 hours after the last application.

FIG. 1 is a diagram representing the cytotoxicity of formulas containing ALE 1833 A, B and C.

As this figure shows, whatever the formula, no significant decrease in the viability of the tissues is observed after treatment.

FIG. 2 is a diagram representing the depigmenting activity of formulas ALE 1833 Band C.

As this figure shows, without EDM, formula ALE1833/B containing kojic acid has no depigmenting activity. With EDM, the same formula has depigmenting activity.

### Conclusion:

EDM can be used to increase the activity of certain active cosmetic molecules, probably through a cutaneous absorption promoter effect.

### EXAMPLE 7

### Example of a Cosmetic Composition Including a Cyclic Acetal from the Invention

### Slimming Cream

| Composition | Quantity (%) |
|---|---|
| Phenoxyethanol, Methylparaben, | 1.0 |
| Butylparaben, Ethylparaben, | |
| Propylparaben | |
| Carbomer | 0.4 |
| Glycerine | 3.0 |
| Xanthan gum | 0.1 |
| Polysorbate-60 | 0.9 |
| Glyceryl Stearate, PEG-100 Stearate | 2.1 |
| Cetyl alcohol | 2.6 |
| Vaseline oil | 7.5 |
| Isopropyl Myristate | 7.5 |
| Alcohol | 5 |
| Cyclic acetal | 5.0 |
| Caffeine | 2.0 |
| Fragrance | 0.2 |
| Triethanolamine | 0.3 |
| Water | to 100.0 |

### Night Cream

| Composition | Quantity (%) |
|---|---|
| Glycerine | 3.0 |
| Propylene Glycol, Diazolidinyl | 1.0 |
| Urea, Methylparaben, Propylparaben | |
| Magnesium Sulphate | 0.7 |
| Cetyl Dimethicone Copolyol | 2.5 |
| Isohexadecane | 5.0 |
| Caprylic/Capric Triglyceride | 5.0 |
| Dimethicone | 5.0 |
| Cyclic acetal | 2.0 |
| Vitamin E acetate | 0.5 |
| Fragrance | 0.1 |
| Water | to 100.0 |

### Self-Tanning Fluid (Microemulsion)

| Composition | Quantity (%) |
|---|---|
| PEG-8 Caprylic/Capric Glycerides | 13.33 |
| Polyglyceryl-6 Dioleate | 8.67 |
| Isostearyl Isostearate | 4.00 |
| Cyclomethicone | 2.30 |
| Diisopropyl Adipate | 1.60 |
| Octyldodecanol | 2.00 |
| PPG-5 Ceteth-20 | 2.00 |
| Phenoxyethanol, Methylparaben, | 0.40 |
| Butylparaben, Ethylparaben, | |
| Propylparaben | |
| Cyclic acetal | 2.00 |
| DHA | 1.00 |
| Water | to 100.00 |

### Anti-Ageing Cream

| Composition | Quantity (%) |
|---|---|
| PEG-30 Dipolyhydroxystearate | 2.4 |
| Isohexadecane | 9.0 |
| PPG-15 Stearyl Ether | 4.5 |
| Caprylic/Capric Triglyceride | 4.5 |
| Magnesium Sulphate | 0.8 |
| Propylene Glycol, Diazolidinyl | 1.2 |
| Urea, Methylparaben, Propylparaben | |
| Cyclic acetal | 2.0 |
| Retinol | 0.5 |
| Poloxamer 407 | 2.0 |
| Glycerine | 3.0 |
| Xanthan gum | 0.7 |
| Fragrance | 0.2 |
| Water | to 100.0 |

### Shampoo (Anti-Dandruff)

| Composition | Quantity (%) |
|---|---|
| Acrylates Copolymer | 1.50 |
| Sodium Lauryl Sulphate | 5.00 |
| Sodium Laureth Sulphate | 4.00 |
| Cocamidopropyl Betaine | 1.50 |
| Polyquaternium-10 | 0.25 |
| DMDM Hydantoin | 0.30 |
| Sodium Hydroxide (20% solution) | 1.30 |
| Citric acid (50% solution) | 0.70 |
| Cyclic acetal | 3.00 |
| Piroctone Olamine | 0.75 |
| Fragrance | 0.50 |
| Sodium Chloride | 0.50 |
| Water | to 100.00 |

1. A pharmaceutical or cosmetic composition comprising:
(a) a cyclic acetal corresponding to the product of the reaction between an aliphatic aldehyde having 2 to 4 carbon atoms and a polyol having 3 to 6 carbon atoms and at least three hydroxyl functions, two of which are located on vicinal carbons or separated by a carbon atom; and
(b) at least one pharmaceutically or cosmetically active ingredient.

2. A pharmaceutical or cosmetic composition according to claim 1, wherein said polyol has 3 to 6 hydroxyl functions.

3. A pharmaceutical or cosmetic composition according to claim 1, wherein said cyclic acetal is the result of the reaction between propionaldehyde and glycerol.

4. A pharmaceutical or cosmetic composition according to claim 1, wherein said cyclic acetal is the result of the reaction between butyraldehyde and glycerol.

5. A pharmaceutical or cosmetic composition according to claim 1, wherein said cyclic acetal is the result of the reaction between acetaldehyde and trimethylolpropane.

6. (canceled)

7. (canceled)

8. (canceled)

9. A method for solubilizing an active pharmaceutical or cosmetic ingredient comprising combining said active pharmaceutical or cosmetic ingredient with a cyclic acetal corresponding to the product of the reaction between an aliphatic aldehyde having 2 to 4 carbon atoms and a polyol having 3 to 6 carbon atoms and at least three hydroxyl functions, two of which are located on vicinal carbons or separated by a carbon atom.

10. A method according to claim 9, wherein said cyclic acetal is the result of the reaction between propionaldehyde and glycerol.

11. A method according to claim 9, wherein said cyclic acetal is the result of the reaction between butyraldehyde and glycerol.

12. A method according to claim 9, wherein said cyclic acetal is the result of the reaction between acetaldehyde and trimethylolpropane.

13. A method for promoting cutaneous absorption of an active pharmaceutical or cosmetic ingredient comprising combining said active pharmaceutical or cosmetic ingredient with a cyclic acetal corresponding to the product of the reaction between an aliphatic aldehyde having 2 to 4 carbon atoms and a polyol having 3 to 6 carbon atoms and at least three hydroxyl functions, two of which are located on vicinal carbons or separated by a carbon atom.

14. A method according to claim 13, wherein said cyclic acetal is the result of the reaction between propionaldehyde and glycerol.

15. A method according to claim 13, wherein said cyclic acetal is the result of the reaction between butyraldehyde and glycerol.

16. A method according to claim 13, wherein said cyclic acetal is the result of the reaction between acetaldehyde and trimethylolpropane.
